(19) United States
(12) Patent Application Publication
(10) Pub. No.: US 2006/0199866 A1
(43) Pub. Date: Sep. 7, 2006

(54) Combination of desoxypeganine and mecamylanine for the treatment of alcohol abuse

(76) Inventors: Joachim Moormann, Werne (DE); Klaus Opitz, Muenster (DE); Hilke Winterhoff, Muenster (DE)

Correspondence Address: D Peter Hochberg;The Baker Building 6th Floor, 1940 East 6th St 6th Floor, Cleveland, OH, 44114, US

(21) Appl. No.: 10/554,350

(22) PCT Filed: Apr. 16, 2004
(86) PCT No.: PCT/EP04/04033

§ 371 Date: Oct. 24, 2005

(30) Foreign Application Priority Data
Apr. 25, 2003 (DE) 103 18714.5

(51) Int. Cl. A61K 31/137

(52) U.S. Cl. 514/649

(57) ABSTRACT

An active substance combination comprising deoxypeganine or of one of its pharmaceutically acceptable derivatives and mecamylamine or one of its pharmaceutically acceptable derivatives. The active substance combination serves to produce a medicament for the treatment of alcohol abuse and/or alcohol dependence.

### CROSS-REFERENCE TO RELATED APPLICATIONS

This application is a National Stage application of International Application No. PCT/EP2004/004033, filed on Apr. 16, 2004, which claims priority of German application number 103 18 714.6, filed on Apr. 25, 2003.

### BACKGROUND OF THE INVENTION

1. Field of the Invention

The present invention relates to pharmaceutical preparations containing 3-deoxypeganine and/or mecamylamine. The invention further relates to the use of this active substance combination for treating the consumption of alcohol, which is detrimental to health, as well as alcohol dependence.

2. Description of the Prior Art

Problem

Of the numerous psychotropic substances with abuse potential, ethanol (in general usage referred to as “alcohol”) is the oldest, the most widely used and the by far most significant in terms of its effects on health and its social and economic consequences. It is assumed that in Germany approximately 1.6 million people are clinically dependent on alcohol, and that 2.7 million consume alcohol on a medically injurious level. About 5 million people must be regarded as being at risk. Every year about 40,000 people—these are by no means only persons clinically dependent on alcohol but also those practicing high-risk consumption of alcohol over extended periods—die each year from the direct consequences of consumption of alcohol. Characteristically, the number of these deaths, as well as that of alcohol cessation therapies, has remained substantially constant in the western industrialized states, although the overall consumption of alcohol has been continuously decreasing for years. This permits the conclusion that the decrease in the overall consumption of alcohol is due above all to wide sections of consumers who have already in the past been relatively health-conscious restricting or foregoing consumption of alcohol, whereas the spreading of high-risk or detrimental consumption of alcohol remains unaltered.

There is thus the task of pharmacologically assisting the reduction of high-risk or detrimental consumption of alcohol—also and particularly of that consumption behaviour which does not yet involve clinical dependence.

State of Science and State of the Art

In European states and/or in the United States of America there are currently five preparations which have approval for use in the drug therapy of alcohol abuse. Of these, bis(diethylthiocarbamoyl)disulfide(disulfiram, ANTABUS®), which has been in use longer than any of the other preparations, has only an aversive effect which does not influence the actual craving for alcohol. Whereas tiapride, a dopamine antagonist operating on the receptor subtypes D2 and D3, has gained little practical significance, the opiate receptor-antagonist naltrexone (REVIA®, DuPont; TREXAN®), and acamprosat (N-acetyl homotaurinate; CAMPRAL®, Merck AG; AOTAL®), which in a complex manner has anti-excitatory action and also influences noradrenergic and dopaminergic pathways, are utilized to a far greater extent, following acute withdrawal, to prevent relapses to abuse of alcohol. Recently, in some European countries the antiexcitatory gamma-hydroxybutyrate (e.g ALCOVER®, Gerot Pharmazeutika) has become available. Naltrexone and gamma-hydroxybutyrate, however, cause considerable gastrointestinal and psychomotoric side effects which impair therapy compliance. In addition, naltrexone is characterized by its low oral bioavailability (approx. 5% of the amount taken in becomes effective) and it is moreover hepatotoxic, whereas gamma-hydroxybutyrate has addiction potential itself.

The long-term success of all the pharmaceutics indicated herein must be regarded as altogether very limited since in the majority of patients they cause an only marginal relapse delay after withdrawal or a clinically insignificant reduction of the amount of alcohol consumed. These medicaments have not had a lasting influence on the fact that on average only 30% of all patients are still abstinent a year after withdrawal treatment.

The therapy of the early stages of a development towards clinical alcohol dependence often spanning several decades (ICD-10 Code F10.2 of the World Health Organization, WHO) and especially the medicinally detrimental consumption of alcohol not yet involving clinical dependence but nevertheless involving high physical and psychiatric potential for damage (ICD-10 Code F 10.1) would, in addition, require medicaments having very few side effects since the so-called “social drinkers”, due to experiencing as yet only little suffering, have hardly any understanding of the problematical nature of their drinking behaviour and therefore show little willingness to suffer such side effects.

Alcohol and all other addiction-producing substances share the ability of activating dopaminergic neurons in the mesolimbic system which represents a central component of the pleasure- and satisfaction-imparting “reward system” in the brain. A dopaminergic therapy may be carried out either via the direct route (by dopamine receptor agonists such as lisuride or bromocriptine) or indirectly by increasing the dopamine concentration locally available in the synaptic gap (e.g. by inhibiting the degradation of the neurotransmitters by monoamine oxidases).

However, the pharmacology of alcohol is complicated, which also finds expression in the above-described diversity of therapeutic approaches. According to current opinion the, on the one hand, sedating and, on the other hand, euphoretic effects and the cognitive- and motor-coordination-impairing effects of alcohol are due to the fact that ethanol shows interactions with the protein subunits of many neuronal receptors and thereby modulates their function. Receptors which represent ion channels are particularly affected by this; in fact they are affected already at concentrations which are by far too low to lastingly impair neuronal membrane structures.

A special position in the therapy of alcoholism which has as yet received little attention is taken up by modulators of cholinergic neurotransmission; these particularly include cholinesterase inhibitors. On the one hand, cholinergically active medicaments are able to enhance the cognition impaired by alcohol-induced damage of the cholinergic pathways and thus increase insight into the problem; on the other hand, cholinergic therapies can also bring about a direct, not cognitively induced reduction in the craving for alcohol. According to current knowledge, this is brought about by the neuronal nicotinic acetylcholine receptors (NACHRs) which are located not only on cholinergic but also on dopaminergic neurons in the mesolimbic system. These receptors are stimulated by an increase in the acetylcholine concentration, and in response thereto release higher amounts of dopamine. They thereby stimulate alcohol-induced dopamine release but without having the effects which alcohol has on other receptors and without causing extremely high dopamine concentrations, so that no significant addiction behaviour is induced. This therapeutic approach could in a wider sense be referred to as partial substitution therapy.

Deoxypeganine (1,2,3,9-tetrahydropyrrollo[2,1-b]chinazoline) is a cholinesterase inhibitor which in pharmacologically relevant concentrations does not bind to NACHRs and which additionally inhibits monoamine oxidase A (but not monoamine oxidase B). This substance is also excellently suitable for the therapy of alcohol abuse, as described by DE 199 06 974 and by the publications WO 00/48600 and EP 1 154 776.

An approach entirely opposite to that of partial substitution therapy is the therapy of substance consumption by blocking the receptor systems which are activated by the respective agonistically active drug of abuse; however, in the case of an existing substance dependence, this therapy can produce withdrawal symptoms which means that there is a high probability of relapse into substance consumption. This applies, for example, to the treatment of nicotine abuse by blocking NACHRs by means of mecamylamine (N-(2,2,3-tetramethyl-bicyclo[2.1.1.]heptane-2-amine).

This racemic mixture of the optical isomers exo-S(+) and exo-R(−)-mecamylamine is an almost 100% orally bioavailable, CNS-penetrant, non-subtype-specific and non-competitive antagonist at neuronal NACHRs which in 1956 was introduced in therapy as an antihypertonic under the trade mark INVERSENE® and INVERSINE®. The two stereoisomers show a differentiated, but essentially comparable behaviour at the individual NACHR subtypes, with the exo-S(+) isomer possibly having a certain selectivity for neuronal NACHRs and thereby reduced peripheral side effects, in particular, on the muscular system. Since mecamylamine in the doses effective for the treatment of essential hypertension of 25 mg/day causes an extensive blockade of the parasympathetic nervous system and thereby leads to an abundance of corresponding side effects, it has been applied only in exceptional cases since 1977. In 2000, mecamylamine was reintroduced in the USA for experimental therapy of certain neuropsychiatric diseases.

U.S. Pat. No. 6,083,962 claims combinations of respective specific antagonists and the substances acting as agonists on respective corresponding receptors and having abuse potential, especially combinations of mecamylamine and nicotine for the therapy of nicotine abuse. This is based on the idea that it should be possible to activate part of the NACHRs by administering nicotine in a pharmacologically suitable, non-addiction-producing form (by an administration form, particularly a transdermal administration form, causing a uniform and controlled release) and thereby satisfy the primary craving for nicotine but prevent the continued consumption thereof by blocking the remaining NACHRs by simultaneously administered mecamylamine. In fact, a synergistic effect of such a fixed active substance combination could be shown in a pilot study, and the effect could even be enhanced by administering mecamylamine singly, prior to smoking cessation (Drug Dev Res 1996; 38:243-56; Exp Clin Phyhopharmacol 1998; 6(3): 33143). According to the results reported in 1998 of three Phase III studies, however, a transdermally administered fixed active substance combination had proved not to be superior to the nicotine patch. However, none of the aformentioned documents addresses the subject of alcohol abuse.

Blomqvist et al, in Eur J Pharmacol 1993; 249(2): 207-13 and Eur J Pharmacol 1997; 334 (2-3): 149-56, teach that mecamylamine completely blocks alcohol-induced increase in extracellular dopamine concentration in the nucleus accumbens of the rat, but without impairing the physiologically significant baseline level of the dopamine release. This is therefore a blockade of the dopaminergic component of the effect of alcohol which in the context of the above described basis is regarded by the authors as an indirect effect mediated by NACHRs. Furthermore, making reference to the above papers as a theoretic basis, Alcohol Clin Exp Res 2002; 26: 326-31 describes a trial on healthy probands who did not exercise alcohol or nicotine abuse. In this study mecamylamine, administered two hours prior to consumption of alcoholic beverages, reduced the centrally stimulating psychotropic effect and presumably also the pharmacokinetics of alcohol. None of these three papers mentions the combination and/or simultaneous administration of mecamylamine with other pharmacologically active substances, in particular, with cholinesterase inhibitors or nicotinic agonists.

The published applications WO 00/35279 and WO 00/35280 claim the two isomers of mecamylamine for the therapy of a plurality of conditions requiring medical treatment, inter alia of alcohol abuse. However, with respect to this option these documents neither indicate biological data nor do they mention any combinations with other pharmacologically active substances for this therapeutic purpose.

### SUMMARY OF THE INVENTION

In light of the above-described state of science, particularly in light of the fact that the pharmacology of alcohol abuse is far more complex than the habit-forming effect of nicotine, a person skilled in the art could by no means assume that deoxypeganine, a substance which acts indirectly on NACHRs due to an increase in the central acetylcholine concentration, would show synergistic action with mecamylamine (a direct inhibitor of NACHRs) with regard to the reduction of alcohol consumption and alcohol preference as compared to non-alcoholic beverages. Surprisingly, this is precisely what is the case.

### BRIEF DESCRIPTION OF THE DRAWINGS

FIG. 1 is a bar graph depicting ethanol preference in female AA rats during the first four and subsequent eight hours after treatment.

FIG. 2 is a bar graph depicting consumption of ethanol in female AA rats four hours and eight hours after treatment.

### DETAILED DESCRIPTION OF THE INVENTION

The subject matter of the invention is thus the combined use of deoxypeganine and mecamylamine to reduce alcohol consumption. Treatment may be performed either by simultaneously administering the two active substances, or by administering mecamylamine singly, immediately followed by a combination of the active substances according to the present invention.

### EXAMPLE 1

### Reduction of Alcohol Consumption and Alcohol Preference in Alcohol-Preferring Rats

The “AA” strain of rats, bred in Finland, has a genetically determined preference for alcohol, which means that even without pre-treatment with alcohol the animals, when given free choice, prefer alcohol-containing liquids, to alcohol-free liquids to satisfy their fluid requirement. This strain has therefore been used in numerous studies on the pharmacology of alcohol and is extremely well characterized.

Female AA rats (tested for alcohol preference and made available by the Public Health Institute in Helsinki) were housed individually and had free access to standard feed (Altromin 1324 granulate), the ambient temperature was 24±1° C. and the light-dark change was 12/12 hours (the dark period lasting from 6 p.m. to 6 a.m. Each cage contained two identical drinking bottles, of which one contained pure water and the other contained aqueous ethanol (10% v/v). During the 12-hour dark period the animals had access to the drinking bottles and during this period had free choice between the two solutions. To prevent the animals from becoming accustomed to a particular position in the cage, the positions of the bottles were changed daily. Prior to start of the tests, the animals were granted an adaptation phase until a largely constant alcohol and water consumption was ensured.

Deoxypeganine hydrochloride (called “DOP” in the following) was obtained from the Institute for the Pharmacology of Plants (Taschkent, Usbekistan) and supplied by the firm of LTS Lohmann Therapie-Systeme (Andemach, Germany) after checking for identity and purity. Mecamylamine was obtained as a commercial preparation from Sigma-Aldrich GmbH (Munich).

Treatment of the test animals always took place immediately prior to the start of the dark period. Mecamylamine was dissolved in 0.9% aqueous saline and a volume of 5 ml/kg body weight was administered by intraperitoneal injection. DOP was applied as an aqueous solution with a volume of 10 ml/kg by a probang.

In the case of combination treatments, this administration took place within a period of less than 10 minutes. Two treatment-free days were always interposed prior to and following the treatment days.

The parameters recorded were consumption of alcohol, consumption of water and consumption of feed (each in grams), as well as alcohol preference, calculated using the formula:

$$Alcohol preference in \% = \frac{( \begin{matrix}consumption of alcohol \text{-} containing \\ drinking solution \times 100\end{matrix} )}{( total consumption of fluid )}$$

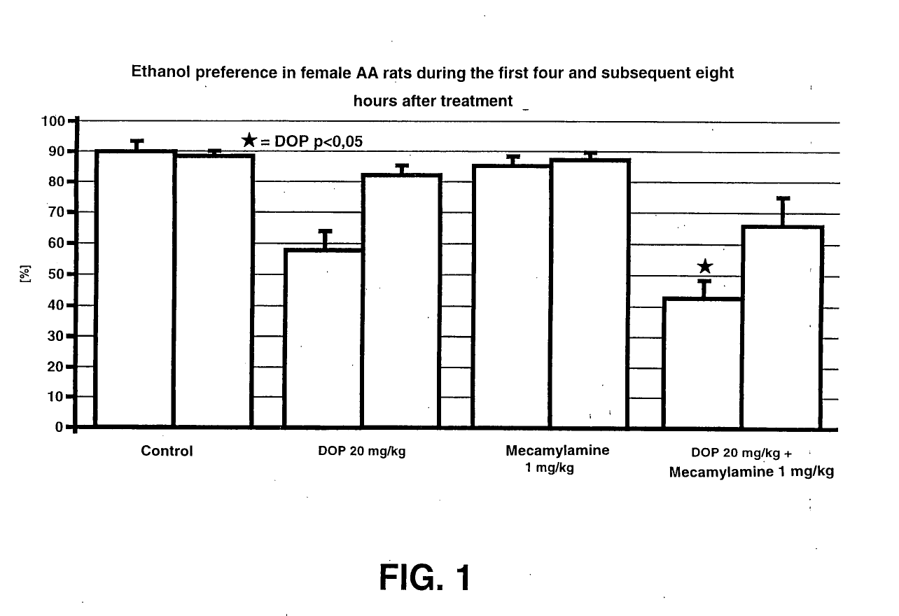

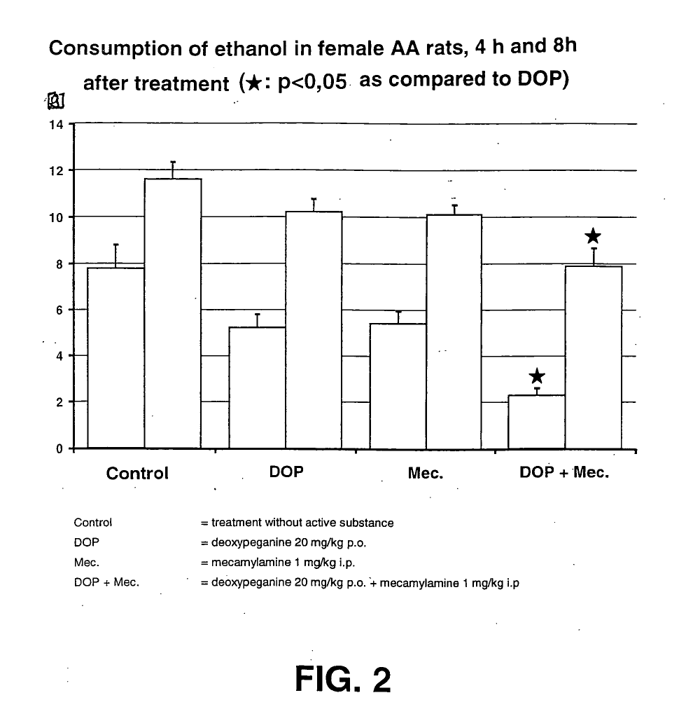

The target parameters were in each case traced during the 12 hours of the dark period following treatment, intermediate results were recorded after the first 4 hours and final results after 12 hours. Statistical evaluation of the test data was performed using the t-test for dependent values. The results in respect of consumption of alcohol and alcohol preference are summarized in FIGS. 1 and 2 as well as in Tables 1 and 2.

TABLE 1
Synergism between deoxypeganine p.o. (DOP) and
mecamylamine i.p. (Mec) in reducing alcohol preference
in female AA rats

| | | ALCOHOL PREFERENCE (%) | | |
|---|---|---|---|---|
| | TREATMENT | After 4 hours | After 8 hours | Total |
| Trial | DOP 20 mg/kg | 57.4 ± 7.1 | 82.0 ± 4.0 | 70.5 ± 4.5 |
| 1 | DOP 20 mg/kg + Mec | 43.3 ± 6.5*) | 66.9 ± 5.7*) | 69.4 ± 5.8*) |
| | 1.0 mg/kg | | | |
| Trial | DOP 20 mg/kg | 55.6 ± 7.6 | 88.0 ± 2.1 | 72.7 ± 4.3 |
| 2 | Mec 1 mg/kg | 85.3 ± 4.2 | 87.8 ± 3.1 | 86.3 ± 2.6 |
| | DOP 20 mg/kg + Mec | 47.2 ± 8.2 | 76.5 ± 6.3 | 66.6 ± 6.8 |
| | 1.5 mg/kg | | | |
| | DOP 20 mg/kg + Mec | 47.7 ± 10.1 | 71.7 ± 6.5*) | 61.1 ± 6.5*) |
| | 1.0 mg/kg | | | |
| | DOP 20 mg/kg + Mec | 54.8 ± 7.7 | 79.6 ± 5.8 | 71.6 ± 5.3 |
| | 0.75 mg/kg | | | |
| | DOP 20 mg/kg + Mec | 59.6 ± 7.3 | 80.9 ± 4.2 | 72.8 ± 4.1 |
| | 0.5 mg/kg | | | |

*)Difference significant (p < 0.05) compared to DOP 20 mg/kg in the respective trial

With peroral administration of 20 mg/kg p.o., DOP lowered the consumption of alcohol and alcohol preference, preferably within the first 4 hours after administration. Mecamylamine (1 mg/kg i.p.) had no effect when administered singly, but potentiated the effect of DOP on both parameters. Low dosages of mecamylamine (0.5, respectively 0.75 mg/kg i.p.) were without effect with regard to alcohol, while the potentiating effect could not be increased further by increasing the mecamylamine dosage to 1.5 mg/kg i.p. (Tables 1 and 2).

TABLE 2
Synergism between deoxypeganine p.o. (DOP) and
mecamylamine i.p. (Mec) in reducing the consumption of 10%
aqueous ethanol
solution in female AA rats.

| | | Alcohol solution consumed (gram) | | |
|---|---|---|---|---|
| | TREATMENT | After 4 hours | After 8 hours | Total |
| Trial | DOP 20 mg/kg | 5.2 ± 0.6 | 10.2 ± 0.6 | 15.4 ± 1.1 |
| 1 | DOP 20 mg/kg + | 2.3 ± 0.3**) | 7.9 ± 0.07**) | 10.2 ± 0.9**) |
| | Mec 1.0 mg/kg | | | |
| Trial | DOP 20 mg/kg | 5.8 ± 0.6 | 10.7 ± 0.5 | 16.6 ± 0.6 |
| 2 | Mec 1 mg/kg | 5.4 ± 0.5 | 10.1 ± 0.4 | 15.5 ± 0.5 |
| | DOP 20 mg/kg + | 2.6 ± 0.4**) | 8.5 ± 0.7**) | 11.1 ± 1.0*) |
| | Mec 1.5 mg/kg | | | |
| | DOP 20 mg/kg + | 2.8 ± 0.5**) | 7.6 ± 0.8**) | 10.4 ± 1.0**) |
| | Mec 1.0 mg/kg | | | |
| | DOP 20 mg/kg + | 3.4 ± 0.6 | 10.6 ± 1.1 | 14.1 ± 1.2 |
| | Mec 0.75 mg/kg | | | |
| | DOP 20 mg/kg + | 3.7 ± 0.5 | 9.9 ± 0.6 | 13.6 ± 0.6 |
| | Mec 0.5 mg/kg | | | |

**)Difference highly significant (p < 0.01 or p < 0.001) compared to DOP 20 mg/kg in the respective trial

Forms of Administration and Treatment According to the Invention

Administration according to the invention may either be in the form of a single medicament with a fixed combination of the two active substances, or be accomplished by administering the active substances in separate forms of administration.

According to the invention the administration of deoxypeganine-HCl may be in the form of tablets or capsules. The daily dose in this case may be 50 to 750 mg, with a daily dose of 100 to 400 mg, which may be divided into an arbitrary number of single doses, being preferred. Furthermore, it is possible to utilise utilize deoxypeganine-containing transdermal therapeutic systems as well as oral and parenteral administration forms with delayed release, as claimed in DE-199 06 974 and the publications WO 00/48600 and EP-1 154 776 derived therefrom, the daily dose being 50-250 mg, preferably administered in a single dose.

According to the invention, the administration of mecamylamine may be performed via the oral route, for instance in the form of the preparation Inversin™ (Targacept, Inc., USA; tablets containing 2.5 mg of racemic mecamylamine hydrochloride); the daily dose may be 2.5-20 mg, with a daily dose of 2.5 to 7.5 mg being preferred. Also usable are transdermal systems or oral administration forms with delayed release formulated according to conventional galenic methods. The daily dose in this case is 0.5-10 mg, preferably administered in a single dose.

According to the invention, the administration of deoxypeganine and mecamylamine may also be performed in the form of medicaments containing fixed combinations of the two active substances which, depending on the mode of administration, are adapted such that the daily dose of deoxypeganine can be 50 to 750 mg and that of mecamylamine 0.5-20 mg.

To those skilled in the art it goes without saying that this enumeration is only by way of example and does not in any way exclude the use of known derivatives of the above-indicated compounds. Thus, in place of the hydrochloride salt of deoxypeganine it is also possible to use its other physiologically tolerable salts or addition compounds, and for certain administration forms the free base, especially for transdermal formulations. Likewise, instead of deoxypeganine one may also utilize the derivatives thereof described in the literature insofar as they are cholinesterase inhibitors. These include 7-bromodeoxypeganine, described in Synthetic Communs. 25(4), 569-572 (1995), 7-halo-6-hydroxy-5-methoxydeoxypeganine, 7-bromo-6-hydroxy-5-methoxydeoxypeganine, 7-chloro-6-hydroxy-5-methoxydeoxypeganine, 7-fluoro-6-hydroxy-5-methoxydeoxypeganine, and 7-iodo-6-hydroxy-5-methoxydeoxypeganine, which are described in Drug Des. Disc. 14, 1-14 (1996), as well as the derivatives of deoxypeganine described in Ind. J Chem. 24B, 789-790 (1985); it is to be borne in mind, however, that above all in the older literature deoxypeganine is frequently referred to under the name of deoxyvasicine.

In the case of mecamylamine, not only the racemate, which is traded e.g. under the name of INVERSINE®, but also each one of the two isomers described in WO 00/35279 and WO 00/35280, also in the form of the respective pharmaceutically acceptable salts and addition compounds, can be used to produce the administration forms according to the invention. The term “salts” is, predominantly but not exclusively, understood to mean the salts of the inventive compounds with halogen acids and with simple organic acids such as tartaric acid (tartrates), succinic acid (succinates), maleic acid (maleates) etc.

Furthermore, according to the invention the above-described treatment with combinations of deoxypeganine and mecamylamine may be preceded by a treatment exclusively with racemic mecamylamine or its individual isomers which is carried through with daily doses of between 0.5 and 20 mg and may last between one day and five days.

The medicament forms utilized according to the present invention to administer a combination of 3-deoxypeganine or of one of its pharmaceutically acceptable derivatives with mecamylamine or with one of its pharmaceutically acceptable derivatives, may contain one or more of the following additives:
anti-oxidants, synergists, stabilisers;
preservatives;
taste corrigents;
solvents, solubilizers;
surface-active agents (emulsifiers, solubilizers, wetting agents, defoamers);
viscosity and consistency-influencing agents, gelling agents;
absorption-accelerating agents;
adsorbents, humectants, lubricants;
disintegration- and solution-influencing agents, fillers (extenders), peptizers; and
release-retarding agents.

This enumeration is not complete; the suitable physiologically acceptable substances are known to those skilled in the art.

The administration of 3-deoxypeganine or one of its pharmaceutically acceptable derivatives with mecamylamine or with one of its pharmaceutically acceptable derivatives may take place via the oral or parenteral route. For oral administration it is possible to produce medicaments in known administration forms such as tablets, coated tablets or lozenges. Apart from these, liquid or semi-liquid administration forms are also suitable; the active substance in this case is present as a solution or suspension. Water, aqueous media or pharmacologically acceptable oils (vegetable or mineral oils) may be used as solvents or suspending agents.

Preferably, the medicaments containing a combination of 3-deoxypeganine or one of its pharmaceutically acceptable derivatives with mecamylamine or one of its pharmaceutically acceptable derivatives are formulated as depot medicaments, which are capable of delivering these active substances to the organism in a controlled manner over an extended period of time.

Moreover, according to the invention the administration of a combination of 3-deoxypeganine or one of its pharmaceutically acceptable derivatives with mecamylamine or one of its pharmaceutically acceptable derivatives can also take place via the parenteral route. To this end, transdermal or transmucosal administration forms can be utilized for the inventive administration of a combination of 3-deoxypeganine or one of its pharmaceutically acceptable derivatives with mecamylamine or one of its pharmaceutically acceptable derivatives to particular advantage, especially adhesive transdermal therapeutic systems (active substance patches). With these, it is possible to deliver the active substance to the patient via the skin, in a controlled fashion and over an extended period of time.

A further advantage is that improper use is more difficult with parenteral application forms than with oral administration forms. Because of the preset active substance-release surface and the predetermined release rate, one can largely exclude overdosage on the part of the patient. In addition, transdermal administration forms are very advantageous because of further properties, e.g. avoiding the first-pass effect or enabling a better, more uniform control of the blood level.

Such transdermal systems containing a combination of 3-deoxypeganine or one of its pharmaceutically acceptable derivatives with mecamylamine or one of its pharmaceutically acceptable derivatives usually comprise an active substance-containing, pressure sensitive adhesive polymer matrix which is covered on the side averted from the skin by an active substance-impermeable backing layer and whose adhesive, active substance-releasing surface is covered with a detachable protective layer prior to application.

The production of such systems and the basic materials and auxiliary materials which may be used in the production are in principle known to those skilled in the art; the structure of such transdermal therapeutic systems, for example, is described in the German patents DE 33 15 272 and DE 38 43 239, or in the U.S. Pat. No. 4,769,028, 5,089,267, 3,742,951, 3,797,494, 3,996,934 and 4,031,894.

As an alternative embodiment of transdermal therapeutic systems in patch form intended for the administration of the inventive active substance combination, so-called reservoir systems may be taken into consideration wherein the active substances are present in a bag which at least on the skin-side consists of a membrane that is permeable to the active substances.

The inventive combination of 3-deoxypeganine or of one of its pharmaceutically acceptable derivatives with mecamylamine or with one of its pharmaceutically acceptable derivatives can be utilized in the therapy of consumption of alcohol which is injurious to health as well as of alcohol dependence in order to reduce the consumption of alcohol.

The inventive combination of 3-deoxypeganine or one of its pharmaceutically acceptable derivatives with mecamylamine or one of its pharmaceutically acceptable derivatives may be utilized for the production of medicaments intended for the therapy of alcohol abuse and/or alcohol dependence, especially to reduce the consumption of alcohol.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

In the claims:

1. An active substance combination comprising deoxypeganine or one of its pharmaceutically acceptable derivatives and mecamylamine or one of its pharmaceutically acceptable derivatives for the production of a medicament for treating alcohol abuse and/or alcohol dependence.

2. The active substance combination according to claim 1, wherein the pharmaceutically acceptable derivative of deoxypeganine is selected from the group consisting of deoxypeganine hydrochloride, 7-bromodeoxypeganine, 7-bromo-6-hydroxy-5-methoxydeoxypeganine, 7-chloro-6-hydroxy-5-methoxydeoxypeganine, 7-fluoro-6-hydroxy-5-methoxydeoxypeganine and 7-iodo-6-hydroxy-5-methoxydeoxypeganine.

3. The active substance combination according to claim 1I wherein the pharmaceutically acceptable derivative of mecamylamine is selected from the group consisting of the salts of mecamylamine with halogen acids and simple organic acids.

4. The active substance combination according to claim 1, wherein mecamylamine is present in a form selected from the group consisting of the racemic mixture of the two stereoisomers of mecamylamine and one of the two stereoisomers of mecamylamine.

5. The active substance combination according to claim 1, wherein the medicament is in the form of a combined administration form for deoxypeganine or one of its pharmaceutically acceptable derivatives and mecamylamine or one of its pharmaceutically acceptable derivatives.

6. The active substance combination according to claim 1, wherein the medicament is in the form of separate administration forms for deoxypeganine or one of its pharmaceutically acceptable derivatives and mecamylamine or one of its pharmaceutically acceptable derivatives.

7. The active substance combination according to claim 1, wherein the medicament is in the form of an administration form to be administered orally or parenterally.

8. The active substance combination according to claim 7, wherein said medicament in the form of a medicament having a depot effect.

9. The active substance combination according to claim 1, wherein an administration form for a daily dose comprises 50 to 750 mg of deoxypeganine or one of its pharmaceutically acceptable salts in the case of an administration form to be administered orally.

10. The active substance combination according to claim 1, wherein an administration form for a daily dose comprises 50 to 250 mg of deoxypeganine or one of its pharmaceutically acceptable salts in the case of an administration form to be administered transdermally.

11. The active substance combination according to claim 1, wherein an administration form for a daily dose comprises 2.5 to 20 mg of mecamylamine in the case of an administration form to be administered orally.

12. The active substance combinations according to claim 1, wherein an administration form for a daily dose comprises 0.5 to 10 mg of mecamylamine in the case of an administration form with delayed release.

13. The treatment of alcohol abuse and/or alcohol dependencies comprising administrating comprising deoxypeganine or one of its pharmaceutically acceptable derivatives and mecamylamine or one of its pharmaceutically acceptable derivatives.

14. A process for treating alcohol abuse and/or alcohol dependence, comprising the steps of administrating an active substance combination comprising deoxypeganine or one of its pharmaceutically acceptable derivatives and mecamylamine or one of its pharmaceutically acceptable derivatives.

15. The process according to claim 14, further comprising the step of pre-treatment with mecamylamine before administrating the active substance combination.

16. The active substance combination according to claim 3, wherein said simple organic acids are selected from the group consisting of tartaric acid, succinic acid and maleic acid.

17. The active substance combination according to claim 7, wherein the medicament is in the form of an administration form to be administered transdermally.

18. The active substance combination according to claim 9, wherein the daily dose of deoxypeganine or one of its pharmaceutically acceptable salts in the case of an administration form to be administered orally is 100 to 400 mg.

19. The active substance combination according to claim 11, wherein the daily dose of mecamylamine in the case of an administration form to be administered orally is 2.5 to 7.5 mg.

20. The process according to claim 15, wherein the pre-treatment step comprises daily doses of between 0.5 and 29 mg of racemic mecamylamine or the individual isomers of mecamylamine and wherein the pre-treatment step lasts between 1 and 5 days.
